(19) United States
(12) Patent Application Publication
(10) Pub. No.: US 2007/0260299 A1
(43) Pub. Date: Nov. 8, 2007

(54) Covalent Grafting of Hydrophobic Substances on Collagen

(75) Inventor: Christian Gagnieu, Chassieu (FR)

Correspondence Address: PAULEY PETERSEN & ERICKSON, 2800 WEST HIGGINS ROAD, SUITE 365, HOFFMAN ESTATES, IL, 60195, US

(73) Assignee: Institut National Des Sciences Appliquees De Lyon, Villeurbanne (FR)

(21) Appl. No.: 11/664,637

(22) PCT Filed: Oct. 3, 2005
(86) PCT No.: PCT/EP05/54972

§ 371 Date: Apr. 4, 2007

Related U.S. Application Data
(60) Provisional application No. 60/615,783, filed on Oct. 4, 2004.

(30) Foreign Application Priority Data
Nov. 5, 2004 (FR) 0411793

(51) Int. Cl. A61K 31/74; A61F 2/00; A61P 41/00; C08H 1/06; A61F 2/16

(52) U.S. Cl. 623/1.1; 424/78.17; 527/101; 623/6.61

(57) ABSTRACT

A hydrophobic grafted collagen, a method for preparing same and use thereof, in particular in therapy, are provided. Hydrophobic substances or molecules are grafted by covalent bonds on reactive amino acid residues of collagen molecules. The chemical linkages serve to modify the physico-chemical and biological properties of collagen and/or its derivatives. In particular, the introduction of hydrophobic residues enable the hydrophilic character of collagen to be modulated and its chemotactic properties involved in cell adhesion and growth to be modified.

The present invention relates to a hydrophobic grafted collagen, its method of preparation and its use, in particular for therapy. In the present invention, substances or molecules of hydrophobic nature are grafted by covalent bonds onto reactive amino acid residues of collagen molecules. The chemical linkages are intended to modify the physicochemical and biological properties of the collagen and/or its derivatives. In particular, the addition of hydrophobic residues allows modulation of the hydrophilic nature of collagen, and modification of its chemotactic properties involved in cell adhesion and growth.

Little or no grafting onto collagen is known in the prior art, aside from the grafting of adhesive peptide onto collagen to increase cell adhesion. Grafting in this case being made using a special chemical process [1]. Some authors also describe collagen grafting onto inert substances such as polyurethane, using processes highly specific to the product and the application [2]. Also mixtures of collagen and fatty acids exist, but no grafting of fatty acids onto collagen, in particular by covalent bonding, is reported in the literature.

Collagen molecules are animal proteins located in the extracellular matrix, which have one or more triple helix domains in their structure. The triple helix is obtained by association of three alpha chains, each consisting of 1050 amino acids. At the end of the chains, non-helical areas of around forty amino acids enable the collagen fibres to bind together. These are telopeptides. These proteins are characterized by their high glycine content (33%) and by the presence of approximately 30% proline and hydroxyproline.

To fabricate and produce biomaterials, collagens of several types and different structural levels are extracted from source tissues by well known methods:
Collagen is said to be native when the entire structure it assumes in the tissues (triple helix and telopeptides) is preserved on extraction.
Collagen can be cleaved enzymatically or chemically at the telopeptides: collagen is then called atelocollagen.
When the three alpha chains of the triple helix are separated by denaturing (e.g. by heating), collagen is said to be denatured.
Regarding gelatin, this is characterized by denaturing of the collagen, and hydrolysis (chemical or thermal) of the alpha chains into peptide fragments.

Different types of collagen have been evidenced, and some have been isolated and industrially produced (essentially Type I and Type IV).

Collagen has varied physicochemical and biological properties making it a material of choice for producing biomaterials. For example, it has specific Theological properties, low antigenicity, plays a role in cell growth and differentiation, and has strong haemostatic properties. In the different areas of medicine, and more particularly in surgery, biomaterials are very frequently used. In general, it is desired to achieve cell adhesion and integration. In recent years however, stress has been laid on developing materials which reduce cell adhesion. Phenomena of post-surgical adherence to biomaterials may occur in addition to the adhesions which are the intrinsic consequences of surgery. Numerous studies are in progress to develop systems making it possible to reduce or eliminate adherence phenomena.

In the prior art, cell adhesion to biomaterials can be reduced by modifying the surface properties of these materials. Surface charge, roughness, exposure of certain chemical structures and hydrophobicity are key factors in regulating cell adhesion. Negative-charged surfaces induce repelling of cells also charged negatively [3,4] and lead to reduced cell adhesion. The roughness of the substrate also plays a key role, since smooth surfaces are anti-adherent. [5,6]. Controlling cell adhesion can also be obtained by grafting chemical or biochemical structures which have a direct influence on molecular events which take place during the interaction of the cells with the materials.

Therefore cell adhesion can be increased by the grafting, onto inert surfaces, of materials known for their adhesion-promoting properties such as collagen [7], hydroxyapatite [5], polylysines [4], hydroxylated polymers or adhesive surface peptides [8].

Similarly, three major strategies exist to reduce cell adhesion:
grafting, onto inert polymers, of bioactive molecules having anti-adhesion properties such as heparin or thapsigargin which directly affect the cell re-organization required for cell adhesion [9,10],
grafting hydrophobic substances such as Teflon [11], polyvinylpyrrolidone or polyacrylamide [12], most generally onto PMMA,
modifying surface hydrophobia using any means, since it has been shown that synthetic polymers, which are increasingly hydrophobic, induce a decrease in cell adhesion [3,6,13,14].

Although collagen is known and used for its adhesion-promoting properties, the subject-matter of the present invention is a novel product having anti-adhesive properties, comprising hydrophobic collagens just as biocompatible as the starting collagen and containing the essential part of the other biological and Theological properties of collagen except its action on cell adhesion and growth. The grafted hydrophobic substances are preferably fatty acids, whether saturated or unsaturated. In relation to the choice of fatty acid used for this grafting, the grafted collagen of the invention is degraded into substances fully recognized by the human body with no pathological reaction.

The present invention therefore concerns a grafted hydrophobic collagen containing fatty acids grafted onto the collagen by covalent bonding. Preferably, the fatty acids are grafted onto the free amine residues of the collagen alpha chain, in particular the free amines of the lysyl residues of the collagen alpha chain.

The percentage of fatty acids, relative to the free amine residues of the collagen alpha chain, lies between 1 and 100%, is preferably higher than around 10% and less than around 85%. According to one preferred embodiment of the invention, the percentage of fatty acids lies between 15 and 50%, and further preferably between 20 and 30%.

The grafted collagen of the invention is a collagen of any origin, in particular a native collagen, a native or denatured atelocollagen, or gelatin. Advantageously, the grafted collagen is a collagen of mammalian origin, preferably porcine, which has advantageously undergone suitable prophylactic treatment to destroy pathogenic agents.

The present invention also concerns a method for preparing a grafted hydrophobic collagen such as defined above and below, in which a suitable quantity of an activated fatty acid is caused to react with the collagen in a suitable reaction medium.

In the grafting method, activation of the carboxylic function of the fatty acid is preferably obtained by forming an activated ester bond or an imidazolide. The activated fatty acid reacts with the deprotonated amines of the lysine epsilon residues of the collagen alpha chains. The activated fatty acid can be either crystallized or prepared extemporaneously in solution.

The activated fatty acid can be obtained by stoechiometric reaction of carbonyldiimidazole (CDI) on the fatty acid in dimethylformamide (DMF) or dimethlylsulfoxide (DMSO). If the activation reaction is made in DMF, the activated product is crystallized and isolated, and then added in solid form to the collagen solution to be grafted. If the activated fatty acid is prepared in DMSO, it is added in solution to the collagen. The preparation of the activated fatty acid in DMF can be used to synthesize all fatty acids between C12 and C22. For all the others, but also for the latter, activation is possible in DMSO. The yield of the activation reaction is greater than 95% and the activated fatty acid shows no measurable loss of activity after 18 months' storage at 4° C. The chemical formula of the activated fatty acid (imidazolide) can be represented by the following formula I: 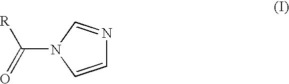
in which R denotes the hydrocarbon chain of the fatty acid.

Activation of the fatty acid may also be achieved by reaction of N-hydroxysuccinimide on the fatty acid preceded by activation with a carbodiimide such as dicyclohexylcarbodiimide or diisopropylcarbodiimide. The activated fatty acid so isolated can be grafted in the same manner as previously onto the collagen. The chemical formula of the activated fatty acid (succinimidyl) can be represented by the following formula II: 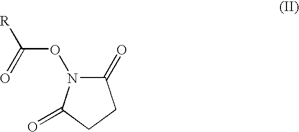
in which R denotes the hydrocarbon chain of the fatty acid.

For the method of the invention, the fatty acid once activated may or may not be isolated from the reaction medium before conducting the grafting reaction.

All the fatty acids can be activated using the above-described means, and can be used in the hydrophobic grafted collagen of the invention and for its method of preparation.

Fatty acids are well known to persons skilled in the art. Fatty acids are aliphatic carboxylic acids containing a hydrocarbon chain of variable length and a carboxyl group (—COOH). The hydrocarbon chain contains more than 6 carbon atoms, generally between 6 and 25 carbon atoms, further preferably between 10 and 22 carbon atoms. The fatty acids can be saturated or unsaturated, containing one or more unsaturations. They may be straight or branched. They may also be substituted by one or more functional groups, in particular functional groups containing one or more oxygen, sulfur or nitrogen atoms, or by one or more halogen atoms. Among the chief linear fatty acids, particular mention may be made of lauric acid (C12), myristic acid (C14), palmitic acid (C16), stearic acid (C18), oleic acid (C18, unsaturated) and linoleic acid (C18, polyunsaturated) or linolenic acid. Lauric acid is the chief component of coco oil (45-50%) and palm oil (45-55%). Nutmeg butter has a high content of myristic acid which accounts for 60-75% of its fatty acid content. Palmitic acid forms between 20-30% of most animal fats, but also of vegetable fats. Stearic acid is the most common of the long chain natural fatty acids, derived from animal or vegetable fat. Finally, oleic acid is the most abundant, natural, unsaturated fatty acid.

The above fatty acids, according to the invention, can be grafted either alone or in a mixture onto the collagen.

Advantageously, the fatty acids are chosen from among stearic, palmitic and myristic acids and their mixtures in any proportion.

Variable, controllable quantities of fatty acids are added by reaction of the activated fatty acid on the lysine residues of the protein. A collagen chain theoretically contains 30 lysine residues. It is therefore possible to graft from 0 to 30 molecules of fatty acids per collagen alpha chain, i.e. a grafting rate of 0 to 100%.

Grafting can be conducted on any type of collagen and irrespective of its structure: native collagen, native or denatured atelocollagen, or gelatin. However, the maximum grafting rates may vary in relation to the lysine content of the collagen under consideration, and to the accessibility of the lysine residues to the reagent, in particular for non-denatured collagens. In relation to the structural level of the collagen to be grafted, different solvents are used such as methanol, dioxan, DMSO or a mixture of solvents in different proportions.

When grafting onto non-denatured collagen, irrespective of the grafting rate and of the grafted fatty acid, grafting is preferably performed on collagen in solution in methanol or in suspension in dimethylformamide (DMF). The previously activated, advantageously crystallized fatty acid as explained above, is added in solution to the collagen in a suitable solvent e.g. a DMF/triethylamine mixture. After reaction, the grafted collagen is precipitated and the precipitate obtained is washed in a suitable solvent, in particular in anhydrous acetone, and dried using usual methods e.g. under reduced pressure.

When grafting onto denatured collagen, irrespective of the grafting rate and of the grafted fatty acid, the collagen is dried overnight under reduced pressure, dissolved and denatured in dimethylsulfoxide (DMSO) at 70° C. In relation to the desired grafting rate, the activated fatty acid is added to the collagen solution under stoechiometric conditions in the presence of a weak base, preferably triethylamine or imidazole, with a view to neutralizing approximately 1.2 mEq H+/g collagen and deprotonating the NH2 functions of the lysine residues. The solution is heated to 60° C. until dissolution of the crystallized, activated fatty acids. The grafting reaction takes place for 16 hours at room temperature. The grafted collagen solutions are then dialyzed against acid water pH 2-3 to remove the DMSO and the bases. The grafted collagen gel obtained is either crushed in 3 volumes dry acetone, then dried under reduced pressure; or melted at 60° C., dried at room temperature and washed in ethyl acetate to remove the residual fatty acids which have not reacted.

For each collagen, the grafting rate is calculated by the difference between the percentage of free amines in the starting collagen, and the percentage of free amines in the grafted collagen. The assay method derives from the work reported by Kakade et al [15]. The quantity of free amines is determined by reaction of 2,4,6-Trinitrobenzene sulfonic acid.

The solubilization of the grafted collagens is conducted in water or a water/ethanol mixture (in different proportions) or in acetic acid. The solvent to be used depends upon the type of fatty acid and the grafting rate. If it is desired to crosslink this collagen in solution, the crosslinking agent is added in an aqueous solution to the grafted collagen solution.

The grafted collagen of the invention may or may not be crosslinked, in particular to produce materials with anti-adhesive properties vis-a-vis living cells. This crosslinking can use conventional crosslinking agents (formaldehyde, glutaraldehyde . . . ) in particular mono-, bi- or polyfunctional reagents and particularly the oxidized, branched polysaccharides (oxidized glycogen and/or oxidized amylopectin for example).

When crosslinking with oxidized polysaccharides, crosslinking of the grafted collagens is obtained by reaction of the aldehyde groups of the oxidized glycogen or oxidized amylopectins with the amines of the lysine residues remaining after grafting onto the collagen. By modifying the ratio of polysaccharide CHO/collagen NH2 from 0.1 to 6, different crosslinking rates can be obtained. Crosslinking takes place by incubating the material obtained after mixing the grafted collagen and the oxidized polysaccharide at pH9, then reducing the remaining aldehyde groups and the formed imine bonds using a reducer (sodium borohydride or sodium cyanoborohydride for example).

With the method of the invention, it is easily possible to obtain a hydrophobic material having anti-adhesive properties, and which can be easily given any suitable form according to intended use. In addition, the choice of fatty acids used and their proportion allow modulation of the anti-adhesive properties of this product. It has been reported in the literature for example that the inhibiting activity of free fatty acids on growth is greater with stearic acid [18-20] than with myristic and palmitic acids [16,17].

After verifying the absence of any indirect toxicity of the grafted collagens, a study of their activity on cell adhesion and growth was conducted on the fibroblastic MRC5 continuous cell line. The same inhibiting activity on growth ascertained for fatty acids alone, was found in the grafted collagens in which the fatty acids are not free. This activity is also more marked for stearic acid (85% inhibition) than for palmitic and myristic acids (65% inhibition). This inhibiting action on cell growth is expressed as soon as the grafting rate reaches 1%, irrespective of the fatty acid. Regarding inhibition of adhesion, an inhibiting activity was also observed on and after a grafting rate of 1%, irrespective of the fatty acid, and it reached a maximum with a rate varying between 20 and 30%.

The present invention also concerns a pharmaceutical or cosmetic composition containing grafted hydrophobic collagen according to the invention, such as defined above and below, in particular an anti-adhesive composition.

The grafted collagen of the invention, regardless of the fatty acid and the grafting rate, can be formed to produce powders, solutions, gels whether crosslinked or not, sponges, granules, films, yarns.

The present invention also concerns an anti-adhesion material containing grafted hydrophobic collagen according to the invention, such as defined above and below.

The composition of these compositions, forms or materials may vary from 0.1 to 100% grafted collagen. Mixtures of grafted collagen with other biopolymers may be prepared e.g. with collagen, atelocollagen, gelatin, glycosaminoglycans, collagens grafted with the same fatty acid but at different grafting rates, collagens grafted with different fatty acids, so as to obtain products having varied physicochemical and biological properties.

When producing a material, whether crosslinked or not, from the grafted collagens, a plasticizer may be added up to a dry matter content of 10%. The plasticizer is preferably glycol, but other products such as lactic acid may also be used.

The grafted collagen of the invention, used alone or in a mixture, can be used in particular:
to produce materials consisting entirely or in part of a collagen modified by covalent grafting of a fatty acid;
to produce materials, sponges, gels, yarns, granules or transparent films of grafted collagen cross-linked using conventional crosslinking agents such as glutaraldehyde and formaldehyde;
to produce materials, sponges, gels, yarns, granules or transparent films of grafted collagen cross-linked with oxidized polysaccharides;
to produce films having a thickness ranging from 20 to 200 μm;
to produce crosslinked, bi-layer films of which one layer consists of collagen irrespective of its non-modified, crosslinked structural level, and the other layer consists of grafted collagen or a mixture of grafted and non-grafted collagen. The grafted collagen also possibly being crosslinked;
to produce composite materials of which one side consists of a sponge of grafted or non-grafted collagen, and one side is formed of a film of grafted collagen or a mixture of grafted and non-grafted collagen;
to produce non-cytotoxic biocompatible materials reducing cell adhesion;
to produce composite materials consisting of a lattice of inert polymers (e.g. polyester, polyurethane) impregnated with a porous or non-porous layer of grafted collagen or a mixture of grafted and non-grafted collagen.

Said collagen, modified by grafting a fatty acid, can be used to produce any biomaterial in which reduced cell adhesion is desired, and in particular to produce materials preventing post-surgical adherence, vascular prostheses or intraocular lenses for example.

The present invention therefore particularly concerns the use of grafted collagens, or a mixture of grafted and non-grafted collagens, to form a material preventing post-operative adherence. The grafted collagen of the invention can therefore be used either alone or in a mixture with other collagens, in particular grafted collagens, to produce single or bi-layer films. It also concerns the association of grafted collagen, or a mixture of grafted and non-grafted collagen, with existing materials such as polymer lattices for example for reinforcement of the abdominal wall. The collagen of the invention may also be used either alone or in a mixture to impregnate such materials.

The present invention also concerns single or bi-layer films so obtained to impregnate lattices with the collagen of the invention.

The invention therefore also concerns a surgical prosthesis, in particular a vascular prosthesis, containing an anti-adhesion material such as defined above and below. It also concerns an intraocular lens containing said anti-adhesion material of the invention.

Finally, the invention concerns the therapeutic use of the hydrophobic collagen such as defined above and below.

The examples of embodiment given below provide illustrations of the invention. Unless otherwise specified, the information on the implementation of examples given below, in particular information on the implementation of methods for preparing grafted collagens according to the invention, may extend to all the above-defined grafted collagens.

The denatured collagen used for the grafting reaction is extracted using a previously described method [21] and is preferably extracted from porcine tissue. During purification, the collagen can be treated with a 1M solution of sodium hydroxide at 20° C. for 1 h with no detectable modification of its chemical structure and biological properties. This treatment is recommended to destroy conventional and non-conventional pathogenic agents [22].

The crosslinking agent, and more particularly oxidized glycogen or oxidized amylopectin, is obtained by periodic oxidation of the polysaccharide in an aqueous medium according to Abdel-Akher et al [23] as modified by Rousseau et al [21]. Determination of the extent of oxidation is performed using a method inspired from Zhao et al [24].

### EXAMPLE 1

### Preparation of Crystallized Stearoyl Imidazolide

To prepare approximately 2.4 g stearoyl imidazolide, 2 g stearic acid are dissolved in 12 ml anhydrous dimethylformamide under heat (40° C.). The reaction being stoechiometric, provision is made to add the quantity of corresponding carbonyldiimidazole with 5% excess, here 1.34 g. In practice, the first half of the quantity of CDI is added to the solution. The crystals of stearoyl imidazolide are precipitated. They are soluble under heat (40° C.). After re-dissolution the remainder of the CDI is added. After 2 hours at room temperature, precipitation of the stearoyl imidazolide crystals is obtained by maintaining the reaction medium at 0° C. for 3 hours. The precipitate is collected by filtering, then washed with 24 ml cold DMF and 12 ml ethanol, and dried. The molecule obtained has a molecular weight of 334.5 g/mol. Its chemical formula is the following:

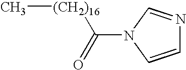

### EXAMPLE 2

### Activation of the Fatty Acid with N-hydroxysuccinimide

Dissolution of 10-20% fatty acid in dioxan at 20° C., then dissolution of N-hydroxysuccinimide (1.3 eq/eq fatty acid) and the addition of cyclohexylcarbodiimide (0.98 eq/eq fatty acid). After reaction for 2 to 3 hours at 20° C., the urea formed by the reaction is removed by filtering, and the filtrate is evaporated to yield an activated ester which is recrystallized in DMF.

### EXAMPLE 3

### Grafting of the Activated Fatty Acid Onto Denatured Collagen: Example of the Grafting of Crystalline Myristoylimidazolide Onto Denatured Atelocollagen, Theoretical Grafting Rate 20%

5 g anhydrous atelocollagen containing 1.6 mmol lysine residues are dissolved in 50 ml anhydrous DMSO at 60° C. 0.322 mmoles (99 mg) myristoylimidazolide (MW 306.5 g/mol) corresponding to 20% of the collagen lysines placed in reaction are added to the collagen solution, and the mixture is heated to 60° C. until dissolution of the crystals of activated fatty acid. 6 mmoles triethylamine are added to deprotonate the lysine epsilon-amine functions, and the medium is left under agitation at 20° C. for 16 hours. On completion of the reaction, the reaction medium is dialyzed against water until total removal of the triethylamine and DMSO. The gel formed during dialysis is melted at 60° C. and the solution obtained is dehydrated under a stream of dry air to yield films. These may be washed in ethyl acetate to extract the fatty acids, whether activated or not, which might not have reacted. The yield lies between 90 and 99%. The grafting rate is determined by assay of the remaining lysine epsilon-amine functions.

The gels formed during dialysis may also be ground in 3 volumes of dry acetone; the grafted collagen is then obtained in powder form.

### EXAMPLE 4

### Grafting of the Activated Fatty Acid Onto Denatured Collagen: Example of the Grafting of Palmitoylimidazolide, with No Isolating of the Imidazolide, Onto Denatured Atelocollagen; Theoretical Grafting Rate 40%

10 g atelocollagen containing 3.2 mmol lysine residues are dissolved in 100 ml anhydrous DMSO at 60° C. 450 mg palmitic acid are dissolved in 1.7% anhydrous DMSO at 60° C. 1.4 mmoles CDI are added to the fatty acid solution. The activation reaction occurs over 2 hours. The crystals of palmitoylimidazolide (320.5 g/mol) are solubilized at 60° C. and the volume corresponding to 1.28 mmol palmitoylimidazolide is added to the collagen solution. The solution is heated to 60° C. until dissolution of the crystals of activated fatty acid. 12 mmoles triethylamine are added to deprotonate the lysine epsilon-amine functions, and the medium is left under agitation at 20° C. for 16 hours. On completion of the reaction, the reaction medium is dialyzed against water until full removal of the triethylamine and DMSO. The gel formed during dialysis is melted at 60° C. and the solution obtained is dehydrated under a stream of dry air to yield films. These may be washed in ethyl acetate to remove the fatty acids, whether activated or not, which might not have reacted. The yield is between 90 and 99%. The grafting rate is determined by assay of the remaining lysine epsilon-amines.

The gels formed during dialysis may also be ground in 3 volumes of dry acetone; the grafted collagen is then in powder form.

### EXAMPLE 5

### Solubilization of the Grafted Collagens with Varying Levels of Stearic, Myristic and Palmitic Acids

All the denatured collagens grafted with stearic, palmitic and myristic acid (with the exception of grafting rates higher than 98%) are heat soluble (60° C.) in a water/ethanol mixture (75:25). To prepare 1 to 2% solutions, the collagen is dissolved in 50% of the final volume in a water/ethanol mixture (50:50). The mixture is heated to 60° C. Once the collagen is dissolved, the medium is diluted to one half with water.

For the denatured collagens with a 98% fatty acid grafting rate, dissolution is only possible in pure acetic acid.

On the other hand, for grafting rates of 30% or less, the denatured collagens are hydrosoluble.

### EXAMPLE 6

### Preparation of Materials Containing Denatured Atelocollagen Grafted with a Fatty Acid. Example of the Crosslinking of Atelocollagen with Oxidized Glycogen

a) Example of the Fabrication of a Film in Culture Plates for Adhesion Tests:

A solution is prepared by mixing solutions of grafted collagen in a concentration ranging from 1 to 2% in a water/ethanol mixture (25:75) with oxidized glycogen at 0.8 moles CHO/mole of saccharide, to obtain a ratio of 2 CHO oxidized glycogen/1 NH2 collagen (21). The collagen solution is obtained by heating to 60° C. until dissolution of the grafted collagen. After cooling, the solution of oxidized glycogen is added and then the glycerol to the proportion of 10% relative to dry matter. 1.5 ml of the end solution obtained are poured into the bottom of wells of a 6-well culture plate. The solution is evaporated under a controlled airflow according to usual, well-known methods for producing films. Crosslinking is obtained by immersing the films in a buffer bath of 0.1M sodium carbonate pH9. The films are washed with distilled water, immersed in a reducing solution of sodium borohydride at 400 mg/L, washed in distilled water, immersed in PBS then dried under a controlled airflow.

b) Example of the Fabrication of Collagen Films 20% Grafted with Stearic Acid and Crosslinked with Oxidized Glycogen at a Ratio of 0.4 Moles CHO/NH2 Mole:

To obtain films 12 cm by 12 cm having a thickness of 45 μm, a 1.75% aqueous solution of collagen 20% grafted with stearic acid is prepared. After cooling, the oxidized glycogen is added to the proportion of 0.4 CHO/NH2 in solution. The glycerol is then added. The end solution is poured onto 144 cm³ polystyrene culture dishes. After gelling, the solution is evaporated under a controlled airflow. Once dry, the films are immersed in 0.1M carbonate buffer pH9 for 45 minutes, washed with distilled water, reduced with a solution of sodium borohydride at 400 mg/l, washed in distilled water, immersed in PBS then dried under a controlled airflow. These films can be sterilized by beta or gamma radiation.

### EXAMPLE 7

### Example of the Fabrication of Collagen Films 30% Grafted with Stearic Acid and Crosslinked with Glutaraldehyde

To obtain films 12 cm×12 cm and 45 μm thick, a 1.75% aqueous solution of collagen 30% grafted with stearic acid is prepared. Glycerol is added to the proportion of 10% collagen dry matter. The end solution is poured onto 144 cm³ polystyrene dishes. The solution, after gelling, is evaporated under a controlled airflow. The dry film is then immersed in a 0.5% glutaraldehyde solution, pH7, for 18 hours, then in a Tris solution, rinsed in PBS and then dried. This film can be sterilized with beta or gamma radiation.

### EXAMPLE 8

### Fabrication of a Freeze-dried Sponge of Collagen 13% Grafted with Palmitic Acid

An aqueous solution of collagen, grafted with 8% palmitic acid, at a concentration of 1 to 2% in water is obtained by heating to 60° C. for 1 hour. The solution is then poured into a metal tub and frozen to −70° C. After 48 hours' lyophilization, sponges are obtained. The mean porosity depends on the collagen concentration and freezing temperature.

### EXAMPLE 9

### Application of the Grafted Collagen to the Fabrication of Implantable Biomaterials

a) In Vitro Cytotoxicity Study

Samples of collagen films grafted with fatty acids such as crosslinked stearic, palmitic and myristic acid, are tested for their cytotoxicity vis-a-vis fibroblasts.

No indirect cytotoxicity was observed, irrespective of the grafting rate of the different fatty acids.

b) In Vitro Study on Cell Growth

Samples of collagen films grafted with fatty acids such as crosslinked stearic, palmitic and myristic acid, are tested for fibroblast growth when in their contact. After 5 days' cell growth, in contact with the films, the cells are separated using trypsin and cell viability is measured by reaction with MTT.

For those films made from collagen grafted with palmitic and myristic acid, irrespective of grafting rate, cell growth is reduced by an average of approximately 65%.

For films made from collagen grafted with stearic acid, irrespective of grafting rate, cell growth is reduced by an average of approximately 85%.

c) In Vitro Study on Cell Adhesion

Samples of collagen films grafted with fatty acids such as crosslinked stearic, palmitic and myristic acid, are tested for fibroblast adhesion in their contact. Separation kinetics with trypsin were performed. In each extract, cell viability was measured by reaction with MTT.

Irrespective of the grafting rate and of the grafted fatty acid, cell adhesion is reduced. Maximum reduction of cell adhesion is observed in the region of a grafting rate of 25 to 30%.

d) In Vivo Study on Biodegradation:

Samples of collagen films grafted with fatty acids such as crosslinked stearic, palmitic and myristic acid are implanted sub-cutaneously in mice.

The biodegradation of the materials in relation to crosslinking rate is studied. Histological studies are used to characterize the host reaction.

Irrespective of crosslinking and grafting rates, no pathological reaction was observed. Mobilization of the immunity system cells is normal. No fibrous shell was observed around the implant.

e) In Vivo Study on Immunogenicity

An immunization protocol for rabbits using crushed collagen 26% grafted with stearic acid was determined.

After 90 days' immunization, no production of antibodies directed against the grafted collagen was evidenced.

### EXAMPLE 10

### Grafting of Stearic Acid Onto Non-denatured Atelocollagen

a) In Methanol

To a homogeneous solution of 500 mg atelocollagen (0.16 mmol lysine) in 30 ml anhydrous methanol, the addition is made of a stearoylimmidazole solution (100 mg, 0.3 mmol) in 5 ml dioxan containing 150 μl triethylamine (1.1 mol). The gel obtained is finely dispersed and the suspension left under agitation at 20° for 24 h. The precipitate is sucked dry and washed with acetone, then dried under reduced pressure.

b) In DMF

To a suspension of 1 g (0.32 mmol lysine) collagen in fine powder in 20 ml DMF, are added 20 ml dioxan containing 200 mg (0.6 mmol)stearoylimmidazole and 300 μl triethylamine (2.2 mmol). After a reaction time of 48 h at 30°, the collagen is collected by filtering, washed with anhydrous acetone and dried under reduced pressure.

### REFERENCES

1. Myles, J. L., B. T. Burgess, and R. B. Dickinson, Modification of the adhesive properties of collagen by covalent grafting with RGD peptides. J Biomater Sci Polym Ed, 2000. 11(1): p. 69-86.
2. Park, J. C., and al., Type I atelocollagen grafting onto ozone-treated polyurethane films: cell attachment, proliferation, and collagen synthesis. J Biomed Mater Res, 2000. 52(4): p. 669-77.
3. Lee, J. H., and al., Interaction of cells on chargeable functional group gradient surfaces. Biomaterials, 1997. 18(4): p. 351-8.
4. Sugimoto, Y., Effect on the adhesion and locomotion of mouse fibroblasts by their interacting with differently charged substrates. A quantitative study by ultrastructural method. Exp Cell Res, 1981. 135(1): p. 39-45.
5. Marois, Y., M. F. Sigot-Luizard, and R. Guidoin, Endothelial cell behavior on vascular prosthetic grafts: effect of polymer chemistry, surface structure, and surface treatment. Asaio J, 1999. 45(4): p. 272-80.
6. Lampin, M., and al., Correlation between substratum roughness and wettability, cell adhesion, and cell migration. J Biomed Mater Res, 1997. 36(1): p. 99-108.
7. Kleinman, H. K., R. J. Klebe, and G. R. Martin, Role of collagenous matrices in the adhesion and growth of cells. J Cell Biol, 1981. 88(3): p. 473-85.
8. Massia, S. P. and J. A. Hubbell, Human endothelial cell interactions with surface-coupled adhesion peptides on a nonadhesive glass substrate and two polymeric biomaterials. J Biomed Mater Res, 1991. 25(2): p. 223-42.
9. Miyata, T., and al., A biodegradable antiadhesion collagen membrane with slow release heparin. ASAIO Trans, 1988. 34(3): p. 687-91.
10. Duncan, G., and al., Thapsigargin-coated intraocular lenses inhibit human lens cell growth. Nat Med, 1997. 3(9): p. 1026-8.
11. Tziampazis, E., J. Kohn, and P. V. Moghe, PEG-variant biomaterials as selectively adhesive protein templates:

model surfaces for controlled cell adhesion and migration. Biomaterials, 2000. 21(5): p. 511-20.
12. DeFife, K. M., and al., Effects of photochemically immobilized polymer coatings on protein adsorption, cell adhesion, and the foreign body reaction to silicone rubber. J Biomed Mater Res, 1999. 44(3): p. 298-307.
13, Werner, L., and al., Evaluation of teflon-coated intraocular lenses in an organ culture method. J Biomed Mater Res, 1999. 46(3): p. 347-54.
14. Altankov, G. and T. Groth, Fibronectin matrix formation by human fibroblasts on surfaces varying in wettability. J Biomater Sci Polym Ed, 1996. 8(4): p. 299-310.
15. Kakade, M. L. and I. E. Liener, Determination of available lysihe in proteins. Anal Biochem, 1969. 27(2): p. 273-80.
16. Legrand, P. and V. Rioux, De l'acide myristique à la myristoylation des protéines. CERIN Cholé-doc, 2000. N^{o} 59.
17. Boutin, J. A., Myristoylation. Cell Signal, 1997. 9(1): p. 15-35.
18. Wickramasinghe, N. S., and al., Stearate inhibition of breast cancer cell proliferation. A mechanism involving epidermal growth factor receptor and G-proteins. Am J Pathol, 1996. 148(3): p. 987-95.
19. Johanning, G. L. and T. Y. Lin, Unsaturated fatty acid effects on human breast cancer cell adhesion. Nutr Cancer, 1995. 24(1): p. 57-66.
20. Singh, R. K., and al., Stearate inhibits human tumor cell invasion. Invasion Metastasis, 1995. 15(3-4): p. 144-55.
21. Rousseau, C. F. and C. H. Gagnieu, In vitro cytocompatibility of porcine type I atelocollagen crosslinked by oxidized glycogen. Biomaterials, 2002. 23(6): p. 1503-10.
22. Dormont, D., [Nature and physicochemical and biological properties of non conventional transmissible agents or prions: consequences for public health]. Pathol Biol (Paris), 1995. 43(2): p. 124-36.
23. Abdel-Akher, M. and F. Smith, Reduction of the products of periodate oxidation of carbohydrates. VII. the constitution of glycogen. Arch Biochem Biophys, 1958. 78(2): p. 451-9.
24. Zhao, H. and N. D. Heindel, Determination of degree of substitution of formyl groups in polyaldehyde dextran by the hydroxylamine hydrochloride method. Pharm Res, 1991. 8(3): p. 400-2.

1. A hydrophobic collagen comprising fatty acids grafted onto collagen by covalent bonding.

2. The hydrophobic collagen of claim 1, wherein the fatty acids are grafted onto the free amine residues of the collagen alpha chain.

3. The hydrophobic collagen of claim 2, wherein the percentage of fatty acids relative to the free amine residues of the collagen alpha chain lies between 1 and 100%, preferably higher than approximately 10% and less than approximately 85%.

4. The hydrophobic collagen of claim 3, wherein the percentage of fatty acids ranges from 15 to 50%, more preferably from 20 to 30%.

5. The hydrophobic collagen of claim 4, wherein the fatty acids are chosen from among stearic, palmitic and myristic acids and their mixtures in any proportion.

6. The hydrophobic collagen of claim 5, wherein the collagen is chosen from among native collagen, native atelocollagen, denatured collagen or atelocollagen, and gelatin.

7. The hydrophobic collagen of claim 6, wherein the grafted collagen is crosslinked.

8. The hydrophobic collagen of claim 7, wherein the grafted collagen is crosslinked with branched, oxidized polysaccharides, chosen in particular from among oxidized glycogen and oxidized amylopectins.

9. A pharmaceutical or cosmetic composition comprising the hydrophobic collagen of claim 8.

10. An anti-adhesive material comprising the hydrophobic collagen of claim 8.

11. A method for preventing post-operative adherence, the method comprising use of the hydrophobic collagen of claim 8.

12. A surgical prosthesis, in particular a vascular prosthesis, comprising the anti-adhesive material of claim 10.

13. An intraocular lens comprising the anti-adhesive material of claim 10.

14. A single or bi-layer film comprising the anti-adhesive material of claim 10.

15. A lattice for reinforcement of abdominal walls, wherein the lattice is impregnated with the material of claim 10.

16. A method for preparing the hydrophobic collagen of claim 8, the method comprising reacting a suitable quantity of an activated fatty acid with the collagen in a suitable reaction medium.

17. The method of claim 16, wherein the fatty acid is activated in the form of an imidazolide or succinimide.

18. The hydrophobic collagen of claim 1, wherein the percentage of fatty acids relative to the free amine residues of the collagen alpha chain lies between 1 and 100%, preferably higher than approximately 10% and less than approximately 85%.

19. The hydrophobic collagen of claim 1, wherein the fatty acids are chosen from among stearic, palmitic and myristic acids and their mixtures in any proportion.

20. The hydrophobic collagen of claim 1, wherein the collagen is chosen from among native collagen, native atelocollagen, denatured collagen or atelocollagen, and gelatin.

21. The hydrophobic collagen of claim 1, wherein the grafted collagen is crosslinked.

22. A pharmaceutical or cosmetic composition comprising the hydrophobic collagen of claim 1.

23. An anti-adhesive material comprising the hydrophobic collagen of claim 1.

24. A method for preventing post-operative adherence, the method comprising use of the hydrophobic collagen of claim 1.

25. A method for preparing the hydrophobic collagen of claim 1, the method comprising reacting a suitable quantity of an activated fatty acid with the collagen in a suitable reaction medium.
